(19) United States
(12) Patent Application Publication
(10) Pub. No.: US 2005/0009114 A1
(43) Pub. Date: Jan. 13, 2005

(54) Identification and quantification of organ-specific proteins derived from human allogeneic cells using proteomics

(75) Inventors: Martin Korbling, Houston, TX (US); Zeev Estrov, Houston, TX (US); Herbert Fritsche, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/846,344

(22) Filed: May 14, 2004

Related U.S. Application Data
(60) Provisional application No. 60/470,915, filed on May 15, 2003.

(51) Int. Cl. G01N 33/53; G01N 33/567; C12N 5/08

(52) U.S. Cl. 435/7.2; 435/372

(57) ABSTRACT

The present invention provides a method for assessing stem cell transplant in a protein-containing sample using multi-dimensional protein separation. Such multi-dimensional separation techniques include chromatography, electrophoresis and mass spectrometry. A protein-containing sample may comprise body fluids such as blood and serum but may also include a cell, tissue or organ. Identification of a protein present in the donor and the transplant recipient but not in the recipient sample before transplant indicates that the stem cell is grafted.

The present application claims the benefit of the filing date of U.S. Provisional Patent Application Ser. No. 60/470,915, filed on May 15, 2003. The entire text of the above-referenced disclosure is specifically incorporated herein by reference without disclaimer.

### BACKGROUND OF THE INVENTION

1. Field of the Invention

The present invention relates generally to the field of stem cell transplant. More particularly, it concerns methods assessing stem cell transplant by identifying donor derived proteins produced by engrafted cells in a transplant recipient, thereby indicating grafting, differentiation and functionality of the stem cell transplant.

2. Description of Related Art

Transplantation of allogeneic and xenogeneic organs, tissues and cells is commonly practiced in humans in order to alleviate numerous disorders and diseases. For example, bone marrow (BM) transplantation is increasingly used to treat a series of severe diseases in humans, such as leukemia. However, transplantation (e.g., bone marrow transplantation) is limited by the availability of suitable donors, since transplanted tissues must traverse major histocompatibility barriers which can otherwise lead to graft rejection. In view of such limitations, approaches for enhancing graft acceptance are needed.

Chimerism is a term used for describing in vivo cells, tissue, organ parts, or entire organs of a genetic constitution that is different from that of the host organism. Hematopoietic chimerism is the best characterized situation of allogeneic donor cells transplanted into a conditioned patient recipient. The recipient's hematopoietic system may be entirely of donor-origin (donor chimerism), entirely of recipient-origin (non-engraftment or graft rejection), or a mixture of donor and recipient elements (mixed chimerism). After allogeneic transplantation, cells, tissues, or entire organs can persist in the host organism, can be lost, or the percentage of donor cells remaining in a particular organ or tissue can vary based on the immunogeneic balance between host and recipient.

Numerous clinical methods have been used and currently are being used to evaluate the origin of hematopoietic cells in the hematopoietic stem cell transplant recipient. Such methods include red blood cell phenotyping, immunoglobulin allotyping, cytogenetic analysis, fluorescence in situ hybridization (FISH), restriction fragment length polymorphism, and mini-satellite or micro-satellite analysis employing polymerase chain reaction (PCR™) techniques. All these techniques evaluate the origin of cells.

On a clinical level, the origin of cells that are part of solid organ tissue following solid organ or hematopoietic stem cell allotransplantation can be evaluated using the Y-chromosome as a marker in a sex-mismatched transplant setting (Körbling et al., 2002; Hematti et al., 2002). Y-chromosome containing cells in female host tissue have been identified by FISH on thin tissue sections (Körbling et al., 2002; Hematti et al., 2002).

Using transplanted hearts from recipients who had died of causes other than graft rejection, Quaini et al. (2002) demonstrated that cells as part of transplanted organ tissue can be replaced by donor-derived cells. In hearts from female donors that had been transplanted into male recipients, approximately 10 percent of the myocytes and coronary arterioles contained a Y chromosome, the definite marker of a male cell. This is compelling evidence of the migration of cells from the recipient into the transplanted heart. Moreover, some of the cells were undergoing division, and others had markers of primitive stem cells. These results indicate that cells from the recipient can enter a graft and contribute to remodeling and growth of the transplanted heart. Whether the migrating cells arose from precursors in the remnants of the recipient's heart tissue or traveled through the recipient's peripheral blood derived from other organs such as bone marrow is not clearly understood.

To study solid organ chimerism, serial tissue sections would have to be analyzed. The interpretation of those data could be hindered by technical conditions. Y-chromosome-positive nuclei must be unequivocally identified as belonging to non-lymphohematopoietic solid organ-specific cells integrated into female tissue, thereby ruling out the possibility that inflammatory donor-derived cells, such as infiltrating lymphocytes or macrophages, are mistakenly identified as solid organ-specific cells (Taylor et al., 2002). Thus, there is need for methods to improve stem cell transplant.

### SUMMARY OF THE INVENTION

Chimerism is a term used for describing in vivo cells, tissue, organ parts, or entire organs of a genetic constitution that is different from that of the host organism. Protein chimerism is known as the presence of both donor and recipient derived proteins in the recipients blood after successful transplantation. Hematopoietic chimerism is a well characterized situation of allogeneic donor cells transplanted into a conditioned patient recipient. A recipient's hematopoietic system may be entirely of donor-origin (donor chimerism), entirely of recipient-origin (non-engraftment or graft rejection), or a mixture of donor and recipient elements (mixed chimerism). Several clinical methods are been used to evaluate the origin of hematopoietic cells in the hematopoietic stem cell transplant recipient. Despite this, new methods are needed to evaluate and improve stem cell transplant.

The present invention therefore provides a method of assessing stem cell transplant comprising (a) obtaining a protein-containing sample from a stem cell transplant recipient; and (b) identifying the presence or absence of a donor stem cell-derived protein in the sample; wherein the presence of a donor stem cell-derived donor protein indicates that the stem cell transplant has grafted.

A protein-containing sample of the present invention may be a body fluid sample such as a blood sample or a serum sample. The blood sample may be a hematopoietic cell sample.

In identifying the presence or absence of a donor stem cell-derived protein in the sample, multi-dimensional protein separation and mass spectrometry may be employed. Multi-dimensional protein separation as contemplated in the present invention may comprise HPLC, ion exchange and/or reversed phase chromatography. In some embodiments of the invention, multi-dimensional protein separation may comprises 2D-gel electrophoresis and isoelectric focusing electrophoresis.

In assessing stem cell transplant a protein-containing sample may be obtained. This may comprise obtaining a pre-transplant sample from a transplant recipient and characterizing proteins in the pre-transplant sample. In some embodiments of the invention, assessing stem cell transplant may further comprise obtaining a protein-containing donor sample and characterizing stem cell-derived proteins from the donor.

In other embodiments of the invention, a protein-containing donor sample may be a fluid, cell, tissue or organ sample. In yet another embodiment of the invention, the donor may be an allogeneic donor having an HLA profile identical to the transplant recipient or an HLA profile not identical to the transplant recipient. In still yet another embodiment, the donor may be an xenogeneic donor.

A transplant recipient or donor as contemplated by the present invention may be a mammal such as a human.

In a particular embodiment of the present invention, obtaining a protein-containing sample from a stem cell transplant recipient (as in step (a) above) may be performed at a time sufficiently post-transplant that donor stem cell-derived proteins from ungrafted stem cells will not be present in the transplant recipient. A time sufficiently post-transplant may be one week or more than one week post transplant. A time sufficiently post-transplant may be about 1 week, about 2 weeks, about 3 weeks, about 4 weeks; or about 2 months, about 3 months, about 4 months, about 5 months, about 6 months, about 7 months, about 8 months, about 9 months, about 10 months, about 11 months, about 12 months; or 1 year or more than 1 year.

A protein-containing cell of the present invention may be a embryonic stem cell, a hematopoietic stem cell, a neuronal stem cell, a bone marrow stem cell, a oral mucosa stem cell, epithelial stem cell, lung stem cell, skin stem cell, gut stem cell, liver stem cell, pancreas stem cell, islet cell stem cell, heart stem cell, muscle stem cell, vascular (endothelial) stem cell, kidney stem cell or mesenchymal stem cell, but is not limited to such.

A protein-containing tissue of the present invention may be from the skin, the liver, the gastrointestinal tract, the kidney, the heart, the blood vessel or derived from the epithelial, mesodermal or endothelial organs, but is not limited to such.

In another particular embodiment, the present invention provides a method of assessing stem cell differentiation following transplant comprising (a) obtaining a protein-containing sample from a transplant recipient; and (b) identifying the presence or absence of a donor differentiated stem cell-derived protein in the sample; wherein the presence of a donor differentiated stem cell-derived donor protein indicates that stem cell transplant has grafted and differentiated.

In a yet another particular embodiment of the present invention, assessing stem cell differentiation following transplant comprise obtaining a protein-containing sample from a stem cell transplant recipient at a time sufficiently post-transplant that differentiation of donor stem cells can occur. A time sufficiently post-transplant may be one week or more than one week post transplant. A time sufficiently post-transplant may be about 1 week, about 2 weeks, about 3 weeks, about 4 weeks; or about 2 months, about 3 months, about 4 months, about 5 months, about 6 months, about 7 months, about 8 months, about 9 months, about 10 months, about 11 months, about 12 months; or 1 year or more than 1 year.

In a yet another particular embodiment of the present invention, there is provided a method of determining tissue site engraftment of a stem cell comprising (a) obtaining a sample from a post-transplant recipient; and (b) assessing the sample for the presence of a tissue selective donor-derived protein in the sample; wherein the presence of a tissue selective donor-derived protein in the sample indicates that the stem cell has engrafted in a tissue site supporting expression of the tissue selective donor-derived protein.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and ate included to further demonstrate certain aspects of the present invention. The invention may be better understood by reference to one or more of these drawings in combination with the detailed description of specific embodiments presented herein.

FIG. 1. Diagram of protein chimerism.

FIG. 2. HPLC spectra before baseline correction. Top row plots are the spectra obtained from patient 1 and displayed as donor, and pre- and post-transplant recipient, from left to right respectively. Bottom row plots are spectra obtained from patient 2 and displayed as donor, and pre- and post-transplant recipient, from left to right respectively. Each spectrum in the plots represent an individual fraction. The unit retention time is in the unit of seconds.

FIG. 3. This illustrates the same set of spectra displayed in FIG. 2, after baseline correction. The spectra is displayed in the same order as in FIG. 2. The retention time is in the unit of seconds.

FIG. 4. Determination of shifting constant between spectra. Top trace is the plot of correlation coefficients vs. index points between the spectra obtained from the donor and the pre-recipient in dataset 2, fraction 6. The shifting constant is the index point that corresponds to the highest correlation coefficient. In this case, it is 131 data points. The bottom trace is plots of uncorrected and corrected spectra. The spectrum indicated by green (obtained from donor) was shifted by 131 data points, so that it matches with the spectrum obtained from pre-recipient (indicated by red).

FIG. 5. Illustration of shifting aligned (interactively) spectra with local adjustment across all three chromatograms. The top spots in each row represent the donor's spectra; the middle spots in each row represent the post-transplant recipient's spectra; and the bottom spots in each row represent the peaks in the pre-transplant recipient's spectra. Each of the boxes represent different spectral regions of the retention time. The size of each spot specifies the peak intensity. The larger size spots represent the higher intensity of the peak.

### DESCRIPTION OF ILLUSTRATIVE EMBODIMENTS

I. The Present Invention

The present invention provides a method of assessing stem cell transplant by obtaining protein-containing samples from a stem cell transplant recipient and a donor, and identifying the presence or absence of the donor derived protein(s) produced by cells engrafted in the recipient.

Previous studies have determined that the same proteins produced by cells of specific organs in different individuals have a slightly different amino acid composition, and therefore are specific to a given individual. The presence of both the donor and recipient derived proteins in the recipient's blood after successful transplantation is termed protein chimerism.

The present invention employs methods of identifying donor stem cell-derived proteins from a body fluid such as blood plasma or serum, from a cell, tissue or organ of a donor and a transplant recipient using a multi-dimensional protein separation technique. This multi-dimensional protein separation employs methods of identifying proteins that are well known to those of ordinary skill in the art and may include but are not limited to various kinds of chromatography such as anion exchange chromatography, affinity chromatography, sequential extraction, and high performance liquid chromatography. Multi-dimensional protein separation may also be accomplished by gel electrophoresis using commercially available reagents and mass spectrometry based methods. Thus, the present invention provides methods to identify and quantify functional human allogeneic donor and recipient cells in human solid organ tissue based on proteomic differences between donor and recipients using proteomic analysis of samples such as peripheral blood samples.

II. Stem Cells

Various tissue-intrinsic adult human stem cells have been described and characterized. These cells are capable of maintaining, generating, and replacing tissue-specific, terminally differentiated cells as a consequence of physiologic cell turnover or tissue damage due to injury. Hematopoietic stem cells (HSCs) trafficking between bone marrow and peripheral blood (PB) are the best-characterized human stem cells. Other stem cell populations may also be contemplated in the present invention including, but not limited to, embryonic stem cells, non-embryonic stem cells such as mesenchymal, neuronal stem cells, and cells derived from any of these; preferably, the stem cell is human stem cell.

The quintessential stem cell is the embryonal stem cell (ES), as it has unlimited self-renewal and multipotent and/or pluripotent differentiation potential, thus possessing the capability of developing into any organ, tissue type or cell type. These cells can be derived from the inner cell mass of the blastocyst, or can be derived from the primordial germ cells from a postimplantation embryo (embryonal gerni cells or EG cells). ES and EG cells have been derived from mice, and more recently also from non-human primates and humans (Evans et al., 1981; Matsui et al., 1991; Thomson et al., 1995; Thomson et al., 1998; and Shamblott et al., 1998).

Stem cells have been identified in most organs and tissues, including “adult stem cells”, i.e., cells (including cells commonly referred to as “progenitor cells”) that can be derived from any source of adult tissue or organ and can replicate as undifferentiated or lineage committed cells and have the potential to differentiate into at least one, preferably multiple, cell lineages. The best characterized are the hematopoietic stem cells. The ultimate hematopoietic stem cell can give rise to any of the different types of terminally differentiated blood cells. This is a mesoderm-derived cell purified based on cell surface markers and functional characteristics (Hill et al., 1996). Also well characterized is the neural stem cell and a number of mesenchymal stem cells derived from multiple sources (Flax et al., 1998; Clarke et al., 2000; Bruder et al., 1997; Yoo et al., 1998; Makino et al., 1999; and Pittenger et al., 1999).

III. Stem Cell Transplantation of Cells Derived From Bone Marrow, Peripheral Blood or Umbilical Cord Blood

Stem cell transplantation (SCT) is being increasingly used in humans. In allogeneic cases (e.g., genetically identical twins) there are no immunological barriers to SCT, but in other circumstances genetic disparities result in immune-related complications, including graft rejection and graft-versus-host disease (GVHD) (Gale and Reisner, 1986). Graft-versus-host disease can be prevented by using T-cell-depleted bone marrow. T-cell depletion of bone marrow may employ any known technique in the art, for example, soybean agglutination and E-rosetting with sheep red blood cells may be employed (Reisner et al., 1980; 1981; 1986).

Allogeneic SCT involves the transfer of allogeneic marrow stem cells from a healthy donor to a patient in need. Following SCT, the patient's bones and hematopoietic niches are reconstituted with donor cells, and the entire hematopoietic system including red blood cells, platelets, nucleated cells, the circulating and tissue-bound reticuloendothelial system and the entire immune system, are converted to be of donor origin (Slavin and Nagler, 1998).

Efficient consistent engraftment of allogeneic stem cells (SC), especially purified stem cells or T cell-depleted stem cells, requires transfer of a large number of stem cells which may be difficult to obtain or are even unavailable (e.g., cord blood stem cells, with limited number of cells; child to adult transplant; etc.; Reisner and Martelli (1995)). Additionally, competition between donor and residual host stem cells for the limited available niches in the bone marrow stroma, as well as the availability of facilitating cells in the donor inoculum may mediate graft failure. This may be overcome by manipulating stem cell competition in favor of donor type cells, by increasing the size of the T-cell depleted stem cell inoculum (Reisner et al., 1978) or by the use of myeloablative drugs such as busulphan, and thiotepa to radiation therapy (Lapidot et al., 1988; Terenzi et al., 1990).

Autologous SCT has shown that, in cancer patients receiving such transplants, treatment with granulocyte colony-stimulating factor (G-CSF) or other cytokines, such as granulocyte macrophage colony-stimulating factor (GMCSF) or interleukin-3 (IL-3), leads not only to elevated levels of neutrophils in the peripheral blood, but also to mobilization of pluripotential stem cells from the marrow to the blood. Thus, peripheral blood stem cells may be obtained after stimulation of the donor with a single dose or several doses of a suitable cytokine, such as granulocyte colony-stimulating factor (G-CSF), granulocyte/macrophage colony-stimulating factor (GM-CSF) and interleukin-3 (IL-3) or any other cytokine as is known to one of skill in the art. In order to harvest desirable amounts of stem cells from the peripheral blood cells, leukapheresis may be performed by conventional techniques (Caspar et al., 1993) and the final product tested for the presence of stem cells. Bone marrow from the donor may be obtained by aspiration of marrow from the iliac crest.

Thus, stem cell transplantation may be used to treat a variety of diseases or disorders including leukemias, such as acute lymphoblastic leukemia (ALL), acute nonlymphoblastic leukemia (ANLL), acute myelocytic leukemia (AML) and chronic myelocytic leukemia (CML), severe combined immunodeficiency syndromes (SCID), osteopetrosis, aplastic anemia, Gaucher's disease, thalassemia and other congenital or genetically-determined hematopoietic abnormalities but is not limited to such.

IV. Obtaining Protein-Containing Samples

In particular embodiments, the present invention contemplates obtaining protein-containing sample such as a fluid, cell, tissue or organ sample. A protein-containing sample of the present invention may be obtained from a donor or a transplant recipient by several means. For example, a blood or serum sample may be obtained by any method as is know in the art. One method of collecting a blood or serum sample may employ venipuncture. Using this method, blood is drawn directly from a blood vessel in the arm of an individual through a needle placed in a single vein. The blood may then be collected in a glass or plastic tube.

A cell, organ or tissue sample of the invention may be obtained by a biopsy. A biopsy is the removal of a sample from the body. Biospies that may be employed in the present invention include punch biopsy or needle biospy but are not limited to such.

A. Punch Biopsy and Cone Biopsy

The present invention contemplates the use of punch or cone biopsy to obtain a protein-containing sample. Punch biopsy is typically used to obtain samples of skin rashes, moles, small tissue samples from the cervix and other small masses. After a local anesthetic is injected, a biopsy punch, (3 mm to 4 mm or 0.15 inch in diameter), is used to cut out a cylindrical piece of skin. The opening is typically closed with a suture and heals with minimal scarring.

Cone Biopsy on the other hand, is used to obtain a piece of tissue which is cylindrical or cone shaped. The advantage of cone biopsy is that it provides a large sample of tissue for analysis.

B. Needle Biopsy

1. Core Needle Biopsy

Core needle biopsy (or core biopsy) is performed by inserting a small hollow needle through the skin and into the organ. The needle is then advanced within the cell layers to remove a sample or core. The needle may be designed with a cutting tip to help remove the sample of tissue. Core biopsy is often performed with the use of a spring loaded gun to help remove the tissue sample.

Core biopsy is typically performed under image guidance such as CT imaging, ultrasound or mammography. The needle is either placed by hand or with the assistance of a sampling device. Multiple insertions are often made to obtain sufficient tissue, and multiple samples are taken. As tissue samples are taken, a click may be heard from the sampling instrument.

Core biopsy is sometimes suction assisted with a vacuum device (vacuum assisted biopsy). This method enables the removal of multiple samples with only one needle insertion. Unlike core biopsy, the vacuum assisted biopsy probe is inserted just once into the tissue through a tiny skin nick. Multiple samples are then taken by using a rotation of the sampling needle aperture (opening) and with the assistance of suction. Thus, core needle biospy or vacuum assisted needle biopsy may be employed in the present invention to obtain a protein-containing sample.

2. Aspiration/Fine Needle Aspiration (FNA) Biopsy

Aspiration biopsy, also referred to as Fine Needle Aspiration (FNA), is performed with a fine needle attached to a syringe. Aspiration biopsy or FNA may be employed in the present invention to obtain a protein-containing sample. FNA biopsy is a percutaneous (through the skin) biopsy. FNA biopsy is typically accomplished with a fine gauge needle (22 gauge or 25 gauge). The area is first cleansed and then usually numbed with a local anesthetic. The needle is placed into the region of organ or tissue of interest. Once the needle is placed a vacuum is created with the syringe and multiple in and out needle motions are performed. The cells to be sampled are sucked into the syringe through the fine needle. Three or four samples are usually made.

Organs that are not easily reached such as the pancreas, lung, and liver are good candidates for FNA. FNA procedures are typically done using ultrasound or computed tomography (CT) imaging.

C. Endoscopic Biopsy

Endoscopic biopsy is a very common type of biopsy that may be employed in the present invention to obtain a protein-containing sample. Endoscopic biopsy is done through an endoscope (a fiber optic cable for viewing inside the body) which is inserted into the body along with sampling instruments. The endoscope allows for direct visualization of an area on the lining of the organ of interest. Samples are obtained by collection or pinching off of tiny bits of tissue with forceps attached to a long cable that runs inside the endoscope of the sample. Endoscopic biopsy may be performed on the gastrointestinal tract (alimentary tract endoscopy), urinary bladder (cystoscopy), abdominal cavity (laparoscopy), joint cavity (arthroscopy), mid-portion of the chest (mediastinoscopy), or trachea and bronchial system (laryngoscopy and bronchoscopy), either through a natural body orifice or a small surgical incision.

D. Surface Biopsy

Surface biopsy may be employed in the present invention to obtain a protein-containing sample. This technique involves sampling or scraping of the surface of a tissue or organ to remove cells. Surface biopsy is often performed to remove a small piece of skin.

V. Multi-Dimensional Separation of Proteins

In order to identify proteins in a protein-containing sample, the present invention employs a multi-dimensional protein separation method that is capable of resolving cellular proteins such as stem cell-derived proteins. A protein separation method as contemplated in the present invention, may employ the use of techniques such as, but not limited to, chromatography, electrophoresis and mass spectroscopy in the identification and quantification of stem cell derived proteins. As used herein, multi-dimensional protein separation refers to protein separation comprising at least two separation steps. In some embodiments, multi-dimensional protein separation refers to two or more separation steps that separate proteins based on different physical properties of the protein (e.g., a first step that separates based on protein charge and a second step that separates based on protein hydrophobicity).

The multi-dimensional protein separation may comprise a first dimension separation of proteins based on a first physical property. For example, proteins may be separated by pI using isoelectric focusing in the first dimension (see, e.g., Righetti, Laboratory Techniques in Biochemistry and Molecular Biology, 1983). However, the first dimension may employ any number of separation techniques including, but not limited to, ion exclusion, ion exchange, normal/reversed phase partition, size exclusion, ligand exchange, liquid/gel phase isoelectric focusing, and adsorption chromatography. In some embodiments (e.g., some automated embodiments). It is preferred that the first dimension be conducted in the liquid phase to enable proteins of the separation step to be fed directly into a second liquid phase separation step.

The second dimension of a multi-dimensional protein separation process may separate proteins based on a second physical property (i.e., a different property than the first physical property) and is preferably conducted in the liquid phase (e.g., liquid-phase size exclusion). For example, some proteins may be separated by hydrophobicity using non-porous reversed phase HPLC in the second dimension (see, e.g., Liang et al., 1996; Griffin et al., 1995; Opiteck et al., 1998; Nilsson et al., 1997; Chen et al., 1994 and 1998; Wall et al., 1999; Chong et al., 1999). This method provides for exceptionally fast and reproducible high-resolution separations of proteins according to their hydrophobicity and molecular weight. The non-porous (NP) silica packing material used in these reverse phase (RP) separations eliminates problems associated with porosity and low recovery of larger proteins, as well as reducing analysis times by as much as one third. Separation efficiency remains high due to the small diameter of the spherical particles, as does the loadability of the reverse phase chromatography columns. However, the second dimension may employ any number of separation techniques. For example, ID SDS PAGE gel may be used. Having the second dimension conducted in the liquid phase facilitates efficient analysis of the separated proteins and enables products to be fed directly into additional analysis steps (e.g., directly into mass spectrometry analysis).

Proteins obtained from the second separation step may be mapped using software in order to create a protein pattern analogous to that of the two-dimensional PAGE image based on the two physical properties used in the two separation steps rather than by a second gel-based size separation technique. A protein profile map as contemplated in the present invention, refers to representations of the protein content of a sample. For example, a protein profile map includes 2-dimensional displays of total protein or subsets thereof expressed in a given cell. Protein profile maps may be used for comparing protein expression patterns (e.g., the amount and identity of proteins expressed in a sample) between two or more samples. Such comparing allows for the identification of proteins that are present in one sample (e.g., a donor sample) and not in another (e.g., recipient cell before transplant), or are over- or under-expressed in one sample compared to the other.

A. Chromatography

Chromatography techniques are well known in the art. These techniques are used to separate organic compounds on the basis of their charge, size, shape, and their solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography include: adsorption, partition, affinity, gel filtration and molecular sieve, and many specialized techniques for using them including column, paper, thin-layer and gas chromatography (Freifelder, 1982).

1. High Performance Liquid Chromatography

High performance liquid chromatography (HPLC) is similar to reverse phase, only in this method, the process is conducted at a high velocity and pressure drop. The column is shorter and has a small diameter, but it is equivalent to possessing a large number of equilibrium stages.

Although there are other types of chromatography (e.g., paper and thin layer), most applications of chromatography employ a column. The column is where the actual separation takes place. It is usually a glass or metal tube of sufficient strength to withstand the pressures that may be applied across it. The column contains the stationary phase. The mobile phase runs through the column and is adsorbed onto the stationary phase. The column can either be a packed bed or open tubular column. A packed bed column is comprised of a stationary phase which is in granular form and packed into the column as a homogeneous bed. The stationary phase completely fills the column. An open tubular column's stationary phase is a thin film or layer on the column wall. There is a passageway through the center of the column.

The mobile phase is comprised of a solvent into which the sample is injected. The solvent and sample flow through the column together; thus the mobile phase is often referred to as the “carrier fluid.” The stationary phase is the material in the column for which the components to be separated have varying affinities. The materials which comprise the mobile and stationary phases vary depending on the general type of chromatographic process being performed. The mobile phase in liquid chromatography is a liquid of low viscosity which flows through the stationary phase bed. This bed may be comprised of an immiscible liquid coated onto a porous support, a thin film of liquid phase bonded to the surface of a sorbent, or a sorbent of controlled pore size.

High-performance chromatofocusing (HPCF) produces liquid pI fractions as the first-dimension of protein separation followed by high-resolution reversed-phase (RP) HPLC of each of the pI fractions as the second dimension. Proteins are mapped (like gels), but the liquid fractions make for easy interface with mass spectrometry (MS) for detailed intact protein characterization and identification (unlike gels) on more selective basis without resorting to protein digestion.

Using HPCF columns, 15-20 total pI fractions-are typically collected over the pH range of 8.5-4.0. Each liquid pI fraction ideally has pI ranges from 0.2 to 0.3 units. These fractions are then analyzed by RP-HPLC to produce high-resolution 2D maps of the expressed proteins present in the sample. Software converts complex chromatograms into easily visualized 2-D maps plotting pI vs retention time (UV signal). These UV pI maps allow for easy comparisons of all intact proteins present in the sample across all the pI fractions. In essence they are pI-hydrophobicity 2D maps.

2. Reversed-Phase Chromatography

In some embodiments of the invention, it is contemplated that multi-dimensional protein separation may comprise reversed phase chromatography. Reversed phase chromatography (RPC) utilizes solubility properties of the sample by partitioning it between a hydrophilic and a lipophilic solvent. The partition of the sample components between the two phases depends on their respective solubility characteristics. Less hydrophobic components end up primarily in the hydrophilic phase while more hydrophobic ones are found in the lipophilic phase. In RPC, silica particles covered with chemically-bonded hydrocarbon chains (2-18 carbons) represent the lipophilic phase, while an aqueous mixture of an organic solvent surrounding the particle represents the hydrophilic phase.

When a sample component passes through an RPC column the partitioning mechanism operates continuously. Depending on the extractive power of the eluent, a greater or lesser part of the sample component is retained reversibly by the lipid layer of the particles, in this case called the stationary phase. The larger the fraction retained in the lipid layer, the slower the sample component moves down the column. Hydrophilic compounds move faster than hydrophobic ones, since the mobile phase is more hydrophilic than the stationary phase.

Compounds stick to reverse phase HPLC columns in high aqueous mobile phase and are eluted from RP HPLC columns with high organic mobile phase. In RP HPLC compounds are separated based on their hydrophobic character. Peptides can be separated by running a linear gradient of the organic solvent.

Along with the partitioning mechanism, adsorption operates at the interface between the mobile and the stationary phases. The adsorption mechanism is more pronounced for hydrophilic sample components while for hydrophobic ones the liquid-liquid partitioning mechanism is prevailing. Thus, the retention of hydrophobic components is greatly influenced by the thickness of the lipid layer. An 18 carbon layer is able to accommodate more hydrophobic material than an 8 carbon or a 2 carbon layer.

The mobile phase can be considered as an aqueous solution of an organic solvent, the type and concentration of which determines the extractive power. Some commonly used organic solvents, in order of increasing hydrophobicity are: methanol, propanol, acetonitrile, and tetrahydrofuran.

Due to the very small sizes of the particles employed as the stationary phase, very narrow peaks are obtained. In some embodiments, reverse phase HPLC peaks are represented by bands of different intensity in the two-dimensional image, according to the intensity of the peaks eluting from the HPLC. In some instances, peaks are collected as the eluent of the HPLC separation in the liquid phase. To improve the chromatographic peak shape and to provide a source of protons in reverse phase chromatography acids are commonly used. Such acids are formic acid, triflouroacetic acid, and acetic acid.

3. Ion Exchange Chromatography

Ion exchange chromatography (IEC) is applicable to the separation of almost any type of charged molecule, from large proteins to small nucleotides and amino acids. It is very frequently used for proteins and peptides, under widely varying conditions. In protein structural work the consecutive use of gel permeation chromatography (GPC) and IEC is quite common.

In ion exchange chromatography, a charged particle (matrix) binds reversibly to sample molecules (proteins, etc.). Desorption is then brought about by increasing the salt concentration or by altering the pH of the mobile phase. Ion exchange containing diethyl aminoethyl (DEAE) or carboxymethyl (CM) groups are most frequently used in biochemistry. The ionic properties of both DEAE and CM are dependent on pH, but both are sufficiently charged to work well as ion exchangers within the pH range 4 to 8 where most protein separations take place.

The property of a protein which govern its adsorption to an ion exchanger is the net surface charge. Since surface charge is the result of weak acidic and basic groups of a protein, separation is highly pH dependent. Going from low to high pH values, the surface charge of proteins shifts from a positive to a negative charge surface charge. The pH versus net surface curve is a individual property of a protein, and constitutes the basis for selectivity in IEC. At a pH value below its isoelectric point a protein (+ surface charge) will adsorb to a cation exchanger (−) such as one containing CM groups. Above the isoelectric point a protein (− surface charge) will adsorb to a anion exchanger (+), e.g., one containing DEAE-groups.

As in all forms of liquid chromatography, conditions are employed that permit the sample components to move through the column with different speeds. At low ionic strengths, all components with affinity for the ion exchanger are tightly adsorbed at the top of the ion exchanger and nothing remains in the mobile phase. When the ionic strength of the mobile phase is increased by adding a neutral salt, the salt ions compete with the protein and more of the sample components are partially desorbed and start moving down the column. Increasing the ionic strength even more causes a larger number of the sample components to be desorbed, and the speed of the movement down the column to increase. The higher the net charge of the protein, the higher the ionic strength needed to bring about desorption. At a certain high level of ionic strength, all the sample components are fully desorbed and move down the column with the same speed as the mobile phase.

Somewhere in between total adsorption and total desorption, the optimal selectivity for a given pH value of the mobile phase is found. Thus, to optimize selectivity in ion exchange chromatography, a pH value is chosen that creates sufficiently large net charge differences among the sample components. Then, an ionic strength is selected that fully utilizes these charge differences by partially desorbing the components. The respective speed of each component down the column is proportional to that fraction of the component which is found in the mobile phase.

Very often the sample components vary so much in their adsorption to the ion exchanger that a single value of the ionic strength cannot make the slow ones pass through the column in a reasonable time. In such cases, a salt gradient is applied to bring about a continuous increase of ionic strength in the mobile phase.

B. Electrophoresis

Gel Electrophoresis techniques are well known to one of ordinary skill in the art. Electrophoresis is the process of separating molecules on the basis of the molecule's migration through a gel in an applied electric field. In an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches an isoelectric point and is thereafter immobile in the electric field. Therefore, this electrophoresis procedure separates molecules according to their different isoelectric points.

Electrophoresis in a polymeric gel, such as a polyacrylamide gel or an agarose gel, adds two advantages to an electrophoretic system. First, the polymeric gel stabilizes the electrophoretic system against convective disturbances. Second, the polymeric gel provides a porous passageway through which the molecules must travel. Since larger molecules will travel more slowly through the passageways than smaller molecules, use of a polymeric gel permits the separation of molecules by both molecular size and isoelectric point.

Thus, electrophoresis in a polymeric gel can also be used to separate molecules, such as RNA and DNA molecules, which all have the same isoelectric point. These groups of molecules migrate through an electric field across a polymeric gel on the basis of molecular size. Molecules with different isoelectric points, such as proteins, can be denatured in a solution of detergent, such as sodium dodecyl sulfate (SDS). The SDS-covered proteins have similar isoelectric points and therefore migrate through the gel on the basis of molecular size. The separation of DNA molecules on the basis of their molecular size is an important step in determining the nucleotide sequence of a DNA molecule.

A polymeric gel electrophoresis system is typically set up in the following way: A gel-forming solution is allowed to polymerize between two glass plates that are held apart on two sides by spacers. These spacers determine the thickness of the gel. Typically, sample wells are formed by inserting a comb-shaped mold into the liquid between the glass plates at one end and allowing the liquid to polymerize around the mold. Alternatively, the gel may be cast with a flat top and a pointed comb inserted between the plates so that the points are slightly imbedded in the gel. Small, fluid-tight areas between the points can be filled with a sample.

The top and bottom of the polymerized gel are placed in electrical contact with two separate buffer reservoirs. Macro-molecule samples are loaded into the sample wells via a sample-loading implement, such as a pipette, which is inserted between the two glass plates and the sample is injected into the well. To prevent sample mixing, it is advantageous to inject-the sample as close to the gel as possible. It is difficult to place the tip of the pipette or loading implement close to the gel because the pipette tip is often wider than the gel.

An electric field is set up across the gel, and the molecules begin to move into the gel and separate according to their size. The size-sorted molecules can be visualized in several ways. After electrophoresis, the gels can be bathed in a nucleotide-specific or protein-specific stain which renders the groups of size-sorted molecules visible to the eye. For greater resolution, the molecules can be radioactively labeled and the gel exposed to X-ray film. The developed X-ray film indicates the migration positions of the labeled molecules.

Both vertical and horizontal assemblies are routinely used in gel electrophoresis. In a vertical apparatus, the sample wells are formed in the same plane as the gel and are loaded vertically. A horizontal gel will generally be open on its upper surface, and the sample wells are formed normal to the plane of the gel and also loaded vertically.

1. Two-Dimensional Electrophoresis

In particular embodiments the present invention employs high-resolution electrophoresis, e.g., one, two-dimensional gel electrophoresis to separated proteins from body fluid or blood serum or a cell, tissue or organ. Preferably, two-dimensional gel electrophoresis is used to generate two-dimensional array of spots of proteins from a sample, which may indicate those proteins involve in stem cell transplantation.

Two-dimensional gel electrophoresis can be performed using methods known in the art (See, e.g., U.S. Pat. Nos. 5,534,121 and 6,398,933). Typically, proteins in a sample are separated by, e.g., isoelectric focusing, during which proteins in a sample are separated in a pH gradient until they reach a spot where their net charge is zero (i.e., isoelectric point). This first separation step results in one-dimensional array of proteins. The proteins in one dimensional array are further separated using a technique generally distinct from that used in the first separation step. For example, in the second dimension, proteins separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of the protein. Typically, two-dimensional gel electrophoresis can separate chemically different proteins in the molecular mass range from 1000-200,000 Da within complex mixtures. The details of this technique are described below.

Two-dimensional electrophoresis is a useful technique for separating complex mixtures of molecules, often providing a much higher resolving power than that obtainable in one-dimension separations. The technique permits component mixtures of molecules to be separated according to two different sets of properties in succession, and lends itself to a variety of different combinations of separation parameters. One combination is separation based on charge followed by separation based on molecular weight, as discussed separately above. Another is separation in a gel of one concentration followed by separation in a gel of the same material but of another concentration. Two-dimensional separations have also been used to create a stepwise change in pH, to separate first in a homogeneous gel and then in a pore gradient gel, to separate in media containing first one molecule solubilizer and then another, or in media containing a solubilizer first at one concentration and then at another concentration, to separate first in a discontinuous buffer system and then in a continuous buffer system, and to separate first by isoelectric focusing and then by homogeneous or pore gradient electrophoresis. Combinations such as these can be used to separate many kinds of molecular components, including serum or cell proteins, bacterial proteins, non-histone chromatin proteins, ribosomal proteins, mixtures of ribonucleoproteins and ribosomal proteins, and nucleic acids.

The first dimension of a two-dimensional electrophoresis system is typically performed in an elongate rod-shaped gel having a diameter in the vicinity of 1.0 mm, with migration and separation occurring along the length of the rod. Once the solutes have been grouped into individual zones along the rod, the rod is placed along one edge of a slab gel and the electric current is imposed across the rod and slab in a direction perpendicular or otherwise transverse to the axis of the rod. This causes the migration of solutes from each zone of the rod into the slab gel, and the separation of solutes within each zone.

Difficulties in two-dimensional electrophoresis arise in the handling of the rod-shaped gel after the first dimension separation has occurred and in placing the gel in contact with the slab gel to prepare for the second dimension separation. The first dimension separation is generally performed while the rod gel is still in the tube in which it was cast. Once the separation in the tube has been performed, the rod is physically removed from the tube, then placed along the exposed edge of the slab gel. The extraction of the rod from the tube and the act of placing it along the slab gel edge require delicate handling, and even with the exercise of great care, the gel is often damaged and the solute zones are distorted or disturbed. Alignment and full contact of the rod with the slab gel are important for achieving both electrical continuity and unobstructed solute migration between the gels. Furthermore, considerable time is involved in the handling and placement of the rod, and errors can result in loss of data. Gel strips can be used as alternatives to the rod, but are susceptible to similar difficulties, opportunities for error, and a lack of reproducibility.

Many of these problems are eliminated by gel packages that contain both the elongated first dimension gel and the slab-shaped second dimension gel in a common planar arrangement that permits the two separations to be done in succession without any intervening insertion or removal of either gel. One such arrangement and method of use is disclosed in U.S. Pat. No. 4,874,490.

A pre-cast gel structure and method has been described in U.S. Pat. No. 5,773,645, which describes a combined water-swellable strip gel and a slab gel on a common support for two-dimensional electrophoresis. In this disclosure, the strip gel is isolated from the slab gel by a fluid-impermeable and electrically insulting barrier. The first dimension separation is performed by placing the liquid sample and buffer in the reservoir to cause the gel to swell and to load it with sample, and then passing an electric current through the reservoir. The barrier, which is joined to the support in an easily breakable manner, is then removed, and the strip gel is placed in contact with the slab gel for the second dimension separation.

In each case, each dimension of the two dimensional electrophoresis is performed in a physically separate gel. When the second dimension is run, the physical discontinuity of the separate gels give rise to a lack of resolution, as well as the need to carefully manipulate the gel during the course of the protocol.

Thus, it would be desirable to provide a gel system and apparatus which would allow the separation of molecules in two dimensions, relying on two separate parameters, within the same gel and not requiring a manipulation or discontinuity to establish and maintain high resolution in each dimension.

An automated system which performs the two dimensional gel electrophoresis in a single gel has been described in PCT Publication WO 96/39625 which utilizes computer controlled robotics to physically rotate the gel slab 90 degrees after the first dimension gel separation has been performed.

An electrophoresis device which eliminates the requirement to physically rotate the gel slab 90 degrees after the first dimension gel separation has been described in U.S. Pat. No. 5,562,813. The device includes an electrophoresis medium enclosed between two plates positioned in contact with a first pair and a second pair of compartments for electrophoresis liquid. Each of the compartments is provided with electrodes to make electrophoretic contact on either side and mutually transversely of each other with the electrophoresis medium, and the compartments are disposed and adapted such that the electrophoresis unit assumes a standing position in the apparatus.

In further embodiments of the present invention proteins in the two-dimensional array can be detected using any suitable methods known in the art. Staining of proteins can be accomplished with colorimetric dyes (coomassie), silver staining and fluorescent staining (Ruby Red). Similar staining for lipids can also be performed. For example, proteins in a gel can be labeled or stained (e.g., Coomassie Blue, Ruby Red, or silver staining). As is known to one of ordinary skill in the art, spots/or protein profiling patterns generated can be further analyzed for example, by gas phase ion spectrometry. Proteins can be excised from the gel and analyzed by gas phase ion spectrometry. Alternatively, the gel containing proteins can be transferred to an inert membrane by applying an electric field and the spot on the membrane that approximately corresponds to the molecular weight of a marker can be analyzed by gas phase ion spectrometry.

C. Isoelectrofusing

In the present invention, it is contemplated that isoelectrofusing may be employed in identifying stem cell derived proteins. By this technique, proteins are extracted from cells using a lysis buffer. To facilitate an efficient process, this lysis buffer should be compatible with that of additional separation and analysis steps to be employed (e.g., reverse-phase, HPLC and mass spectrometry) in order to allow direct use of the products from each step into subsequent steps. Such a buffer is an important aspect of automating the process. Thus, the preferred buffer should meet two criteria: 1) it solubilizes proteins and 2) it is compatible with each of the steps in the separation/analysis methods. One skilled in the art can determine the suitability of a buffer for any particular configuration by solubilizing a protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

After extraction in the lysis buffer, proteins are initially separated in a first dimension. The proteins are isolated in a liquid fraction that is compatible with subsequent techniques (reverse phase HPLC) and mass spectrometry steps. n-octyl β-D-glucopyranoside (OGI, from Sigma) may be used in the buffer. This is one of the few detergents that is compatible with both reverse-phase chromatography and HPLC and subsequent mass spectrometry analyses.

After extraction, the supernatant protein solution is loaded to a device that can separate the proteins according to their pI by isoelectric focusing (IEF). The proteins are solubilized in a running buffer that again should be compatible with reverse phase HPLC. A suitable running buffer is 6 M urea, 2 M thiourea, 0.5% n-octyl β-D-glucopyranoside, 10 mM dithioerythritol and 2.5% (w/v) carrier ampholytes (3.5 to 10 pI).

D. Mass Spectrometry

In some embodiments of the present invention, the proteins of the second separation step are further characterized using mass spectrometry. For example, the proteins that elute from the chromatography separation are analyzed by mass spectrometry to determine their molecular weight and identity. For this purpose the proteins eluting from the separation can be analyzed simultaneously to determine molecular weight and identity. A fraction of the effluent is used to determine molecular weight by either matrix-assisted laser desorption ionization (MALDI-TOF-MS) or electrospray spectrometry (ESI) or time-of-flight (TOF) (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI or TOF. The molecular weight 2D protein map is matched to the appropriate digest fingerprint by correlating the molecular weight total ion chromatograms with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

Separated proteins may be analyzed by mass spectrometry to facilitate the generation of detailed and informative 2D protein maps. The nature of the mass spectrometry technique utilized for analysis in the present invention may include, but is not limited to, ion trap mass spectrometry, ion trap/time-of-flight mass spectrometry, quadrupole and triple quadrupole mass spectrometry, Fourier Transform (ICR) mass spectrometry, and magnetic sector mass spectrometry. Applications of mass spectrometric methods are well-known to those of skill in the art and are discussed in Methods in Enzymology, 1990.

Various MS techniques can be used to further analyze the subfractions for detailed identification and characterization of the proteins. Moreover, the second dimension can run directly to an MS, whereby both the UV/pI maps as well as the mass/pI maps for the intact proteins can be obtained using the software to display both. Having the mass analysis of the intact proteins allows for direct comparison with the matrix-assisted laser desorption ionization (MALDI) peptide mass mapping analysis of the protein to observe differences between the intact molecular weight (MW) and the database MW values.

Advances in one-dimensional capillary separation techniques based on size, charge, or hydrophobicity directly-coupled to ITIS have been critical in narrowing the gap between high throughput genomic and proteomic methodologies. Separation methods and NIS analysis have become more automated and sensitive. The current generation of mass spectrometers can fragment and analyze peptides at speeds of several hundred per hour. Combined with progressive improvements in reliability and affordability, these factors have propelled the mass spectrometer to the forefront of proteomics research. Key developments include peptide ionization methods such as electrospray ionization (ESI) and matrix-assisted laser desorption/ionization (MALDI). ESI uses a voltage placed across a fine needle to create a mist of fine droplets of charged particles. MALDI co-crystallizes the protein/peptide of interest in a matrix designed to absorb laser energy at a specific wavelength. A laser is then used to excite the matrix, causing ionization of the protein. Both of these techniques are amenable to automation. When combined with quadropole orthogonal acceleration time-of-flight (QTOF) mass analyzers, MALDI can provide analytic sensitivity to the sub-femtomolar level. This improved sensitivity allows detection of low-abundance proteins and significantly reduces the amount of tissue needed for analysis.

MS measures the charge-to-mass ratio of an ionized protein or peptide fragment. Mass spectrometers have been used to identify specific proteins with a known mass extraction from two-dimensional electrophoresis gels. However, because proteins are usually too large to be analyzed directly by MS, the protein or spot excised from a gel can be proteolytically digested into smaller peptide fragments. The mass of each of these peptides can be measured in the spectrometer, creating a profile of component peptide masses which, when compared to the known mass of the undigested protein, define a “peptide mass fingerprint” characteristic for a specific protein. A protein can be identified by comparing its peptide mass fingerprints with fingerprints produced by in vitro digestion of every protein in a database.

A significant improvement to the 2D electrophoresis/MS fingerprint method is the direct analysis of peptide sequences by tandem mass spectrometry (HIS/MS). The key feature of this method is the ability of a tandem mass spectrometer to collect amino acid sequence information from a specific peptide, even if many other peptides are concurrently present in the sample. Here, a peptide ion of interest is isolated from other peptide ions in the spectrometer and passed into a collision cell where it undergoes further fragmentation through collision with an inert gas (collision-induced dissociation, CID), breaking the peptide randomly at each peptide bond. The resultant peptide fragment masses create a unique spectrum that can determine the sequence of the parent peptide. In ESI, liquid chromatography is used as a separation technique, producing a steady stream of peptides from the digestion of a complex mixture of proteins that are delivered continuously to the mass spectrometer for identification. Both simple and complex protein mixtures can then be analyzed e.g., the protein mixture can be a multiprotein receptor complex, such as the T-cell receptor, or a subcellular domain, such as the membrane fraction of a population of cells. As in protein mass mapping, a search algorithm is then applied, and the masses of every sequence of consecutive amino acids in the database are compared to the experimental fragment masses. Despite the generation of hundreds of thousands of peptide fragments, the probability of a false match is low, and the probability of matching the masses of every amino acid between two different peptides is also low. From-the sequence of the peptide, the identity of a protein is determined by correlating the CID spectrum with the contents of sequence databases.

Clearly the most important prerequisite for protein identification by MS is the presence of the protein sequence and peptide mass fingerprint in available databases. Predicted protein sequence data derived from EST and genomic databases has greatly accelerated the automation of the identification process.

VI. Comparative Proteomics: Isotope-Coded Affinity-Tags

Because the proteome is in a dynamic state, comparative proteomics requires a quantitative, systematic, and global analysis analogous to the use of microarray technology in the study of the transcriptome. A recently developed technique, called the isotope-coded affinity tag (ICAT) method, can measure the relative expression level of proteins in a complex protein derived from two differentially labeled cell populations. The ICAT reagent is a molecule with three functional domains: a biotinylated tag, a linker sequence containing either 8 deuterium atoms (heavy reagent) or 8 hydrogen atoms (light reagent), and a cysteine-reactive group. Similar to the use of differential fluorescent dye labeling in cDNA microarray analysis, proteins from one cell population are labeled with the heavy reagent and those from the other are labeled with the light reagent. After treatment with the ICAT reagents, equal quantities of each protein sample are combined. At this point, any fractionation technique can be used to reduce the complexity of the starting mixture or enrich for low-abundance proteins. The fractions are then digested with trypsin and the ICAT-labeled peptides are isolated by avidin-biotin affinity chromatography. These peptides are then analyzed by microcapillary LC NIS/MS. Tandem MS is used first to analyze the paired atomic masses for each peptide (light vs. heavy peptides) and then, after further fragmentation, the amino acid sequences are determined. The relative intensities of the differently tagged forms of a peptide are proportional to their relative abundance. The isotropic substitutions in ICAT reagents do not affect the biophysical properties; the only difference due to the ICAT tag is 8 mass units for singly charged peptides and the two tagged peptides elute at different times. Thousands of peptides can then be identified and their relative abundance determined, allowing a global view of protein abundance in cells or tissues in two different states in a single experiment. The success of these methods in rapidly characterizing large numbers of proteins present in complex mixtures has been demonstrated in prostate and human myeloid leukemia cancer cell lines (Sechi, 2002; Gygi et al., 2002; Zhou et al., 2002; Turecek, 2002).

Several features of the ICAT method make it suitable for the automated, quantitative, and Global analysis of the proteome. By selectively labeling only cysteine-containing peptides, the complexity of the peptide mixture is reduced approximately 10-fold without significant reductions in protein quantification or identification. The quantification and identification of proteins with multiple cysteine-containing digestion fragments add redundancy to the analysis. The ICAT alkylation reaction can be performed in the presence of protein-stabilizing reagents such as urea, sodium dodecyl sulfate (SDS), and salts that enhance sample integrity, and the peptide samples eluted from the avidin-affinity column require no further purification before analysis by LC MS/MS. Recently, ICAT labeling methods have been improved by the development of a solid-phase isotope labeling reagent. This solid phase isotope tagging method is simpler, more efficient and more sensitive, and amenable to automation.

ICAT methods provide a broadly applicable means for quantitative comparison of protein expression in a variety of normal and disease states, a task that is critically important for the identification of antigenic targets in immunotherapy. Of particular interest is the adaptation of this method for identification and characterization of membrane proteins indicative of neoplastic transformation. A recent report describes the identification and quantification of 491 microsome-associated proteins expressed in human myeloid leukemia (HL-60) cells before and after induction of differentiation with 12-phorbol 13-myristate acetate (PMA) (Jackson et al., 2001) The isolation and separation of membrane-associated proteins are refractory to 2DE techniques because of their hydrophobicity and poor solubility. In this study, Han et al. (2001) developed a protocol for the isolation of microsomal fractions from HL-60 cells using differential ultracentrifugation before protein labeling with ICAT reagents, multidimensional chromatography, and automated tandem MS. Whereas microarray analysis might identify changes in expression of genes associated with the microsomal fraction by computational methods, the direct proteomic analysis positively identifies proteins associated with the membrane and can suggest post-translational modifications such as acylation, prenylation, and protein-protein interactions.

A significant challenge remains in the development of methods to quantitate post-translational modifications on a global scale. The addition of initial enrichment steps in an analysis targeted at protein phosphorylation can provide a rich substrate for further automation using the ICAT method (Gygi et al., 2002; Han et al., 2001; Zhou et al., 2002; Turecek, 2002) New reagents and methods are being developed to extend this approach to other biochemical modifications and protein-protein interactions.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedure

Blood samples were obtained from the donor prior to the collection of stem cells from peripheral blood and from the recipient at two times: pre-transplant and post-transplant. The pre-transplant blood sample was taken prior to chemotherapy and transplantation and the post-transplant sample was taken at least 4 weeks after stem cell transplant, when there was evidence of hematopoietic engraftment of the donor by the recipient.

The blood samples were analyzed by two dimensional high performance liquid chromatography (2D-HPLC) and protein maps were obtained. These protein maps were compared, and specific proteins that were unique to the donor, and not present in the pre-transplant recipient sample were found in the post-transplant recipient blood sample. These proteins were obtained from the column eluates, and identified by N-terminal sequencing performed by mass spectrometry. The presence of certain proteins such as enzymes demonstrate the functionality of the transplanted cells.

### Example 2

### Protein Chimerism of the Stem Cell Transplant

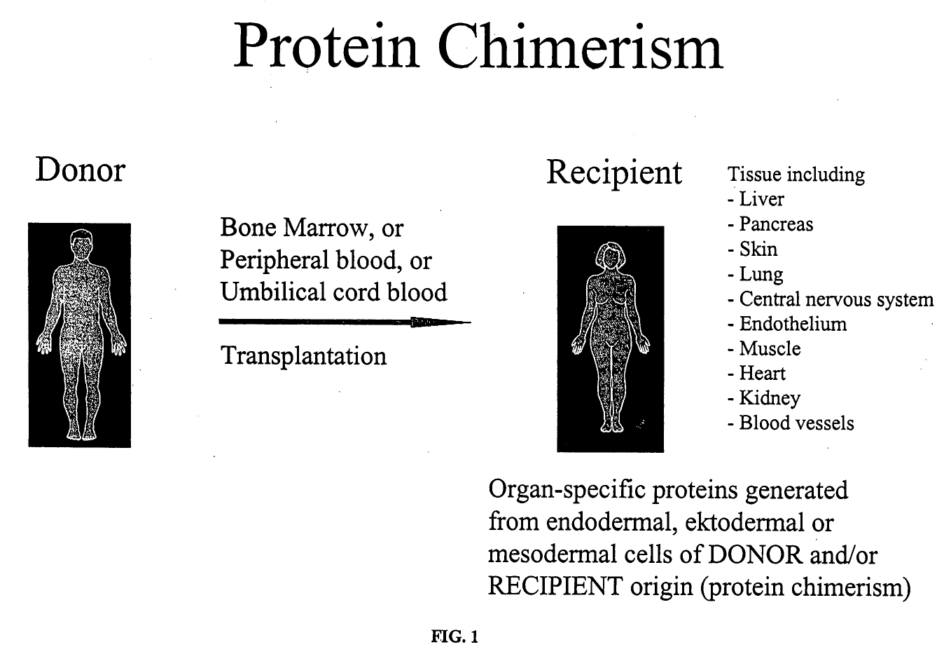

The presence of both the donor and recipient derived proteins in the recipient's blood after successful transplantation is known as protein chimerism (FIG. 1). Studies are conducted to demonstrate the protein chimerism resulting from the successful engraftment and functionality of the stem cell transplant. Stem cells are obtained from peripheral blood, bone marrow, umbilical cord blood and other human tissues representing hematopoietic and non-hematopoietic tissues. The multi-dimensional protein separation procedure described herein is used to analyze the protein samples.

### Example 3

### Assessing Stem Cell Transplant in Various Tissues

A donor-derived stem cell injected into a recipient may differentiate in various organs, such as the liver or kidney, but nonetheless maintain its functionality. Thus, studies are conducted to demonstrate the successful engraftment and functionality of stem cell transplant in multiple target tissues of the recipient. As described in Example 1, blood samples are collected and multi-dimensional protein separation techniques are used to analyze the protein products which identified that tissue grafting had occurred.

### Example 4

### Detecting Stem Cell Transplant Between Different Genders

Blood samples can be collected as described in Example 1 above, and multi-dimensional protein separation techniques described herein, used to detect proteins derived from transplanted stem cells that are isolated from male or female donors. This study is used to demonstrate successful differentiation into recipient cells of the opposite gender and which could produce the appropriate sex hormones.

### Example 5

### Analysis of HPLC Data

The following example describes the inventors' attempts to prepare the raw data obtained according to the HPLC methods described in the above Examples and elsewhere in the application, in a graphical form. This graphical presentation is intended to provide a more straightforward way to view the data, but is not limiting in any way, nor is it required for the practice of the invention. As discussed below, there are a number of considerations that must be taken into account when moving to this sort of data display.

The present invention examines the effects of stem cell treatments in serum sample obtained from patients based on change of proteins signature in HPLC chromatograms between the pre-transplant recipient and the post-transplant recipient. The donor's serum samples were also collected and the HPLC measurement performed; the donor samples were used as the reference sample. Table 1 summarizes the results obtained.

TABLE 1
Description of HPLC Spectral Data

| | | Number | | |
|---|---|---|---|---|
| | | of Frac- | Descrip- | |
| Dataset | Spectra Series | tions | tion | Patient |
| Dataset | SC_1_11512829 | 15 | Donor | 1 |
| 1 | SC#2_11512830 | 15 | Pre/ | 1 |
| | | | recipient | |
| | SC#7_080802_11512830 | 15 | Post/ | 1 |
| | | | recipient | |
| Dataset | SC_#4_11512830 | 15 | Donor | 2 |
| 2 | SC#3_080702_11512830 | 15 | Pre/ | 2 |
| | | | recipient | |
| | SC#20_080902_11512831 | 15 | Post/ | 2 |
| | | | recipient | |
| Normal | SC#10_11512829 | 15 | female/ | 1 |
| | | | Normal | |
| Female | SC11_082702_11512832 | 15 | female/ | 2 |
| | | | Normal | |
| | SC12_082902_11512832 | 15 | female/ | 3 |
| | | | Normal | |
| | SC#13_090602_11512833 | 15 | female/ | 4 |
| | | | Normal | |
| | SC#14_090502_11512833 | 14 | female/ | 5 |
| | | | Normal | |
| Normal | SC9_LB treated | 15 | male/ | 1 |
| | | | Normal | |
| Male | SC#15_090502_11512833 | 15 | male/ | 2 |
| | | | Normal | |
| | SC#16_090902_11512833rev | 15 | male/ | 3 |
| | | | Normal | |
| | SC#17_091002_11512833rev | 15 | male/ | 4 |
| | | | Normal | |
| | SC#18_091002_11512833rev | 15 | male/ | 5 |
| | | | Normal | |

The study involved two pair datasets (dataset 1 and 2 as indicated in Table 1). Each set involved three samples: donor, pre-recipient, and post-recipient. Each of these samples contains 15 fractions. The fractionations were separated based on isoelectric point (p.I). The data also contain HPLC spectra contributed from 10 normal populations, 5 females and 5 males. A total of 239 chromatograms were analyzed. The analysis focused only on the dataset 1 and dataset 2.

In conducting this study, the inventors identified peaks present in serum samples obtained from the post-transplant recipient and the donor, but absent in the serum sample obtained from the pre-transplant recipient. The emphasis was given to the smallest peaks which meet this criteria. Below is a detailed description of the procedure used and the analysis of the results obtained.

Data Preprocessing

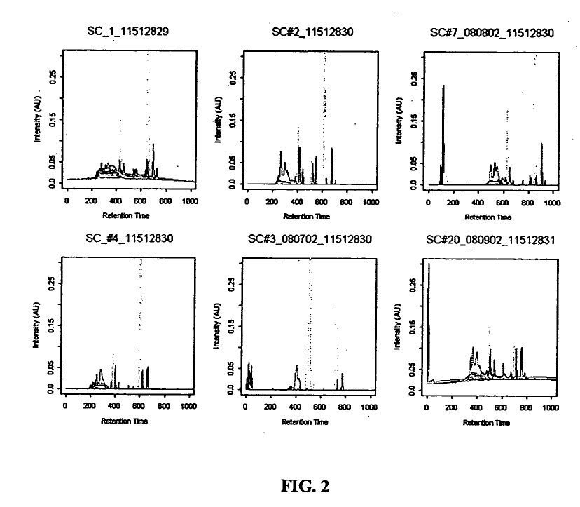

The original spectra exhibited non-uniform baseline (FIG. 2). In order to accurately carry out the data analysis, baseline corrections were required at the first stage of the analysis.

A simultaneous peak detection and baseline correction procedure was performed. This step was carried out on each spectrum using an algorithm that was developed in-house and implemented in MATLAB (The MathWorks, Inc., Natick Mass.). Briefly, the algorithm first estimates potential peak locations by finding all local maxima in the spectrum and eliminating obviously spurious peaks using a series of heuristic criteria (such as the distance to the nearest local minimum must be greater than a global noise estimate; the slope from the maximum to the local minimum must exceed half the noise).

To carry out baseline correction, peaks are first temporarily removed from the spectrum, and the baseline is estimated by fitting a monotone local minimum in a fixed window (20 minutes wide along the retention time axis) from left to right across the spectrum. The peaks are then placed back, and the baseline is subtracted, the entire process is repeated. The algorithm produces a list of retention times where peaks are located along with a baseline-corrected spectrum.

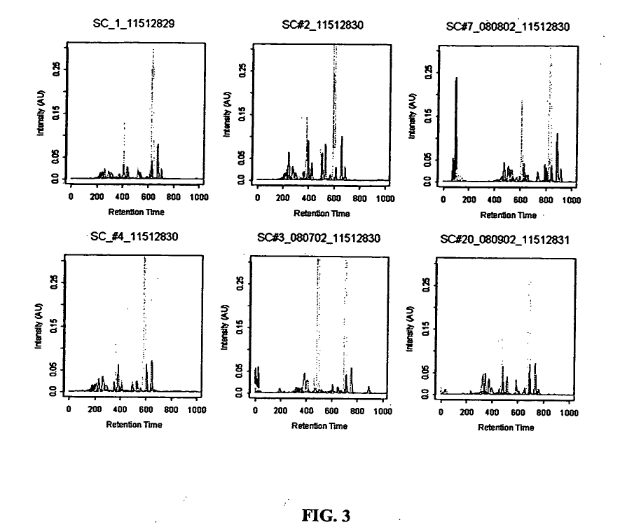

The spectral baseline correction for each of the 239 spectra was carried out using the same criteria. FIG. 2 illustrates the set of spectra from dataset 1 and 2 (all fractions) before baseline correction, and FIG. 3 demonstrates the same set of spectra after performed baseline correction.

The local signal-to-noise (S/N) ratio of each peak was also computed by dividing its height by the median absolute deviation from the median in a window centered at the peak. The signal-to-noise ratio is used to filter the peaks more sensibly in a later step of the analysis.

Two major problems were noted from the display of the baseline-corrected spectra. These were normalization and alignment. The first problem is that the spectra were not being normalized. The intensity contributed from the same protein component varies in a large range between spectra, i.e., the same peak in two different spectra has different intensities (in some spectra this is very different). This proposed a problem in distinguishing “small changes” between (across) spectra. The second problem is critical; it was found that the peaks' positions (retention times) within the same fraction obtained from pre-recipient, post-recipient and donor are not exactly matched. The differences between two samples in the same fraction vary from a few seconds to over a few ten seconds, or even more in retention time. This problem was exhibited in all fractions of both datasets, as well as in all chromatograms collected from normal populations.

It was assessed that this problem might be the result from different experimental parameter settings in various experiments performed on different days. This problem may be overcome by performing calibration experiment for each set of spectra. Thus, several algorithms were used to calibrate these spectra.

Algorithms for Aligning Two Chromatograms

In order to resolve the misalignment problem, two algorithms were applied as described below.

Shifting spectrum by a constant. In the first approach one spectrum was shifted by a constant value to match another (reference spectrum). This is the simplest solution to the problem. This approach assumes that the two mismatched chromatograms are off by a constant in the entire spectral region, and therefore it could be corrected by shifting one chromatogram with that constant to match another. Properly chosing the shifting constant is crucial.

A statistical approach was used to determine the shifting constant for each paired spectra based on the correlation coefficients in a defined region within the two spectra. Briefly, the method computes correlation coefficients for each point between two spectra within a spectral region (contains 400 index points). To illustrate this finding, the calculated correlation coefficients against the index points were plotted from −400 to 400. The index point corresponding to the maximum correlation coefficient is the shifting constant between the two spectra. In other words, a spectrum was shifted to match another spectra based on the highest correlated point between the two spectra. This method was applied to the dataset.

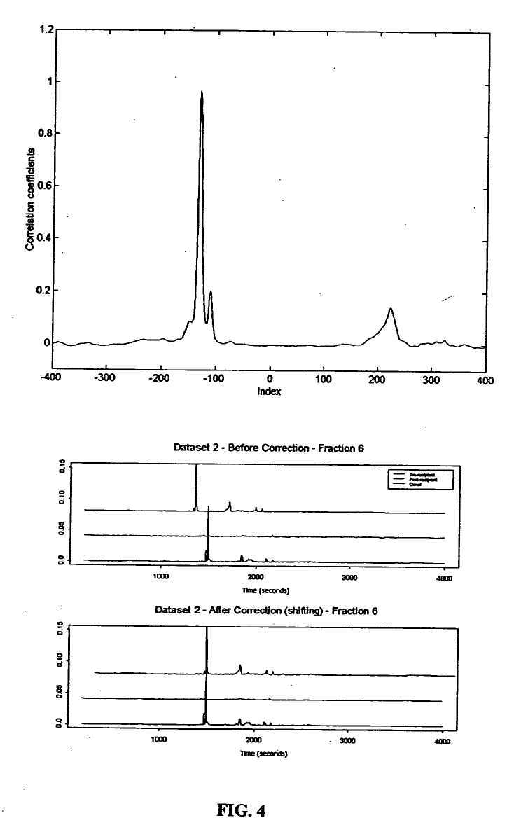

The alignments between spectra were improved in some fractions and was demonstrated using the three spectra in dataset 2, fraction 6 (FIG. 4). The index point was plotted from negative to positive, the negative and positive indexes indicate the shifting direction.

It was noted in the alignment between two spectra that not every single point in the two spectra aligned. More importantly, this approach could potentially produce false alignment. The correlation coefficients were computed based on peak intensities in a defined spectral region. Because the spectra were not normalized, peak intensities contributed from the same protein components might not have the same intensity (or even close to being the same). In this case, the highest correlation might not correspond to the same peak contributed from the same biological component in the two spectra, and the alignment between spectra would be inaccurate.

Shifting spectrum interactively. Next the inventors attempted to shift the spectra interactively. This approach was based on the use of commercial software Grams/32°, produced by Thermo Galactic. The software provides multiple functions for spectral manipulations such as derivative, baseline correction, peak fitting, etc. It also allows shifting of two spectra interactively so that the two spectra can be matched together. This interactive shifting alignment is based on visual observation of good overlapping peaks. This software was applied to the datasets.

Briefly, a spectrum that needs to be aligned (the adjusting spectrum) was first selected and then a reference spectrum (the adjusting spectrum aligned to) was chosen. The software allows the adjusting spectrum to be moved freely on x-axis (the retention time) until the two spectra matched. In the current datasets, the spectra of a pre-recipient from dataset 1 was chosen as the reference spectrum on each fraction of both datasets, and every spectrum was adjusted to the reference. This approach is easy to use, and it does not change the spectral feature (intensity and band shape).

Mathematical robust alignment approaches such as quadratic polynomial smooth warping developed by Paul Eilers (Leiden University Medical Center) were also used. In addition, a linear warping alignment approach developed in house was also used. However, none of these methods produced ideal alignment solution.

Comparing all the approaches disclosed herein, it was decided that the interactive shifting alignment algorithm would be used for the analysis. Although the alignments between spectra were improved using this approach, it was found that not all of the peaks in two spectra can be exactly matched. This suggests that misalignment of two spectra is not constant for each data point, and cannot be aligned perfectly. This was found to be true in all the approaches used.

Once the spectra were aligned, they were exported into Matlab and Splus2000 (a statistical software) for analysis.

Data Analysis

In order to identify peaks detectable in post-recipient and donor's spectra but not detectable in pre-recipient's spectra, several steps to identify potentially “significant” peaks were used.

Peak filtering: A detection filter was used to decide which peaks should be retained for further analysis. In this analysis, a peak was retained only if it met the condition of a signal-to-noise ratio, S/N>2. The general intent is to only retain peaks that meet a certain “believability” threshold, which can happen either because the signal stands out well above the noise or because a noisier peak can be identified multiple times. The condition of S/N was set at a low value so that the small peak feature would be remaining (since the changes in chromatograms might be potentially small). Using this criterion, roughly about 90-120 peaks in each fraction of both datasets were identified.

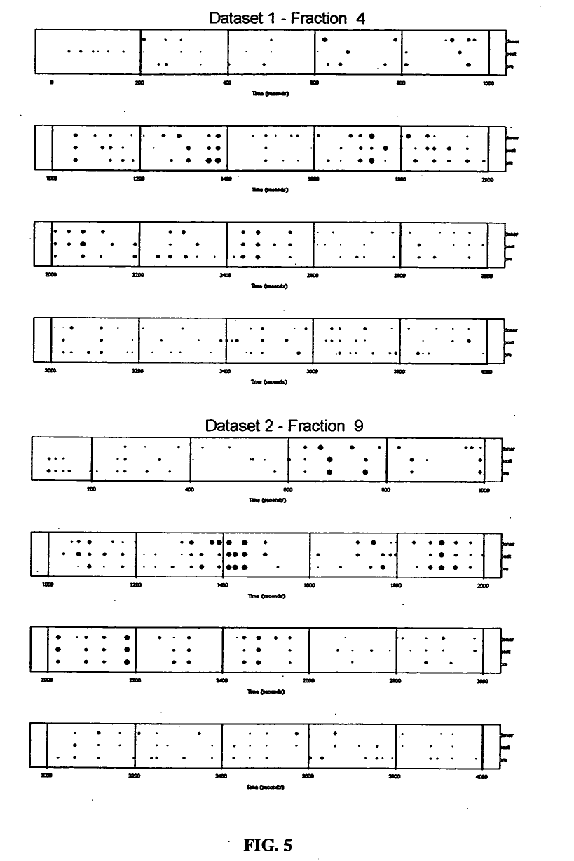

FIG. 5 demonstrates a new graphic method to visualize peaks that passed filtering criterion. In the plots, the peaks identified on each spectrum (based on the signal-to-noise ratio) are displayed as a spot. The size of the spot indicates the intensity of the peak; large size spot represents higher intensity.

Local Adjusting Peak Alignment

As discussed above, the alignment approach can be used to match the major features of two spectra; however it cannot perfectly align two spectra point-by-point. It appears that the identified peaks across three samples did not match precisely even after alignment in all fractions. Local adjustments are needed in order to match peaks exactly so that identifying significant changes in spectral features across three spectra are feasible. Thus, a procedure was developed for adjusting identified peaks that still misalign across spectra.

In the process, the differences in retention time between identified peaks for each pair of spectra, reference (pre-recipient), and the one need to be adjusted (post-recipient and donor) was first computed. The smallest difference between peaks, i.e., to find the closest pair of peaks in both spectra was then determined.

If the difference between the pair of closest peaks in both spectra were less or equal to a “window” size (length of 15 seconds), the peak position (retention time) of the adjusting spectrum by the nearest peak's position of the reference spectrum was restored. This “window” was used to check and correct each paired closest peaks in both spectra. Through this process, the peaks in the adjusting spectrum were slightly adjusted so that they matched with the peaks in the reference spectrum.

It is noted that false alignments are associated with this adjustment. The selection of “window” size is critical. The larger the size of the window selected the better the alignment produced, but it also associated with the high possibility of false alignment in certain regions. This is especially true when there exist multiple peaks that are close to each other within the window region. Selecting a small size of “window” could avoid such a problem, but is not sufficient enough to adjust misalignment peaks. Several lengths of the “window,” 5, 10, 15, 20 seconds, were tested in an attempt to eliminate the false alignment rate as much as possible. A length of 15 seconds “window” was chosen. This “window” was applied to both datasets for peak local alignment.

Applying this process improved the alignments (FIG. 5). However, it is noted that false alignments cannot be completely avoided. Thus, the goal is to minimize false alignments as much as possible.

Identifying Peaks in the Three Spectra

The purpose of this study is to identify peaks present in the samples obtained from the post-transplant recipient and the donor, but absent in the pre-transplant recipient. In nature, these peaks are expected to be small or appear as a shoulder of high intense peaks. Therefore, the focus was on identifying peaks that existed in donor, and in post-recipient with low intensity, but not presented pre-recipient. The following criteria was used to emphasize the above conditions: 1) Peaks only found in post-recipient spectra and donor's spectra; 2) Peaks in post-recipient spectra having an S/N<5 (small peaks); and 3) Peaks in the donor spectra having an S/N>5. Using these criteria, a number of peaks were identified from each fraction on both datasets. These peaks are listed in Table 2. The table lists peaks found between 200 and 3000 seconds retention time.

TABLE 2
Peaks present in post-transplant recipient & donor, not present in pre-
transplant recipient
(In the region 200-3000 seconds)

| | DataSet 1 | | | | | |
|---|---|---|---|---|---|---|
| | | | Peak | Dataset 2 | | |
| | Reten- | | S/N | Reten- | | |
| | tion | | (Post- | tion | | Peak S/N |
| | time | Peak S/N | recip- | Time | Peak S/N | (Post- |
| Fraction | (sec) | (Donor) | ient) | (Sec) | (Donor) | recipient) |
| 1 | 575* | 7.55 | 4.38 | 420 | 19.39 | 4.69 |
| | 1342 | 6.59 | 3.13 | 1793 | 27.62 | 4.72 |
| 2 | 2043 | 20.36 | 2.36 | 1207 | 5.77 | 2.69 |
| | | | | 2143 | 16.09 | 3.21 |
| 3 | 293* | 7.65 | 2.15 | 1776 | 26.67 | 4.39 |
| | | | | 2087 | 248.26 | 2.40 |
| 4 | 208* | 44.18 | 2.69 | 1210 | 7.66 | 2.33 |
| | 500* | 6.34 | 3.63 | 2160 | 11.49 | 2.55 |
| 5 | 309 | 5.82 | 3.04 | | | |
| | 380 | 7.21 | 2.40 | | | |
| | 1464 | 5.63 | 4.47 | | | |
| | 2990 | 5.34 | 4.10 | | | |
| 6 | | | | | | |
| 7 | 482 | 9.64 | 4.91 | 2200 | 24.08 | 2.70 |
| | 1266 | 30.35 | 4.13 | | | |
| 8 | | | | | | |
| 9 | | | | 1306 | 5.68 | 2.15 |
| | | | | 2909 | 9.80 | 4.50 |
| 10 | 575 | 14.79 | 2.63 | | | |
| | 1020 | 6.05 | 2.16 | | | |
| 11 | 2809 | 5.08 | 2.56 | 210 | 14.05 | 3.15 |
| | | | | 2534 | 9.07 | 2.79 |
| 12 | 2798 | 6.83 | 3.86 | 228 | 55.64 | 2.16 |
| | | | | 1545 | 17.41 | 4.83 |
| 13 | 383 | 7.90 | 2.08 | 247 | 22.25 | 3.11 |
| 14 | 610 | 10.80 | 3.19 | 405 | 18.01 | 2.22 |
| 15 | 445 | 9.36 | 2.61 | 739 | 10.68 | 4.39 |
| | 1134 | 8.70 | 3.30 | 1807 | 6.86 | 2.07 |
| | 2829 | 5.01 | 3.81 | | | |
| | 2952 | 5.81 | 2.25 | | | |
| 16 | 2433 | 20.90 | 2.30 | 2918 | 6.59 | 4.59 |

Since the beginning part of each spectrum is dominated by a high intensity peak not contributed from the biological sample, this part of the spectrum (0-200) was omitted. The nature of the peaks in the region 200 to 3000 are sharper, and peaks in the region 3000 to 4000 are broader and associated with certain degree of noise.

To overcome some of the problems encountered in conducting this analysis, experimental calibration and normalization will be employed in future experiments.

As can be seen, there are a number of issues that must be taken into account when preparing the data for graphical presentation, including baseline correction, normalization and alignment. Ultimately, this approach may not prove to be the optimal way for presenting and analyzing data. However, it clearly may be applied as a “first pass” approach for identifying relevant peaks, thereby highlighting certain fractions for further analysis by other methodologies (e.g., mass spectroscopy). Again, it is emphasized that this type of data processing is not required for the practice of the invention as described herein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,874,490
U.S. Pat. No. 5,534,121
U.S. Pat. No. 5,562,813
U.S. Pat. No. 5,773,645
U.S. Pat. No. 6,002,127
U.S. Pat. No. 6,398,933
Bruder et al., J. Cell. Biochem., 64:278-294, 1997.
Caspar et al., Blood, 81(11):2866-2871, 1993.
Chen et al., Rap. Comm. Mass Spec., 12:1994, 1998.
Chong et al., Rap. Comm. Mass Spec., 13:1808, 1999.
Clarke et al., Science, 288:1666, 2000.
Evans et al., Nature, 292:154-156, 1981.
Flax et al., Nature Biotechnol., 16:1033-1039, 1998.
Freifelder, In: Physical Biochemistry Applications to Biochemistry and Molecular Biology, 2nd ed. Wm. Freeman and Co., NY, 1982.
Gale and Reisner, Lancet., 1(8591):923-926, 1988.
Griffin et al., Rap. Comm. Mass Spec., 9:1546, 1995.
Gygi et al., J. Proteome Res., 1(1):47-54, 2002.
Hans et al., Nat. Biotechnol., 19(10):946-951, 2001.
Hematti et al., Experi. Hematol., 30:943-949, 2002.
Hill et al., Exp. Hematol., 24:936, 1996.
Jackson et al., J. Clin. Invest., 107:1395-1402, 2001.
Körbling et al., New Eng. J. Med., 346:738-746, 2002.
Lapidot et al., J. Immunol., 141(8):2619-2624, 1988.
Liang et al., Rap. Comm. Mass Spec., 10:1219, 1996.
Makino et al., J. Clin. Invest., 103:697-705, 1999.
Matsui et al., Nature, 353:750-752, 1991.
Methods in Enzymology, In: Mass Spectrometry, McCloskey (Ed.), Academic Press, NY, Vol. 193, 1990.
Nilsson et al., Rap. Comm. Mass Spec., 11:610, 1997.
Opiteck et al., Anal. Biochem., 258:344, 1998.
PCT Appl. WO 96/39625
Pittenger et al., Science, 284:143, 1999.
Quaini et al., New Engl. J. Med., 346:5-15, 2002.
Reisner and Gan, Cancer Res., 45(9):4026-4031, 1985.
Reisner and Martelli, Immunol. Today, 16(9):437-440, 1995.
Reisner et al., Lancet., 2(8208-8209):1320-1324, 1980.
Reisner et al., Lancet., 2(8242):327-331, 1981.
Reisner et al., Proc. Natl., Acad., Sci., USA, 75(6):2933-2936, 1978.
Righetti, In: Laboratory Techniques in Biochemistry and Molecular Biology, Work et al. (Eds.), Amsterdam, p. 10, 1983.
Sechi, Rapid Commun. Mass Spectrom., 16(15):1416-1424, 2002.
Sharnblott et al., Proc. Natl. Acad. Sci. USA, 95:13726, 1998.
Slavin and Nagler, Ann. Oncol., 9(1):S31-S39, 1998.
Taylor et al., Circulation, 106:2-4, 2002.
Terenzi et al., Transplantation, 50(40):717-720, 1990.
Thomson et al., Proc. Natl. Acad. Sci. USA, 92:7844-7848, 1995.
Thomson et al., Science, 282:1145-1147, 1998.
Turecek, J. Mass Spectrom., 37(1):1-14, 2002.
Wall et al., Anal. Chem., 71:3894, 1999.
Yoo et al., J. Bone Joint Surg. Am., 80:1745-1757, 1998.
Zhou et al., Nat. Biotechnol., 20(5):512-515, 2002.

1. A method of assessing stem cell transplant comprising:
a) obtaining a protein-containing sample from a stem cell transplant recipient; and
b) identifying the presence or absence of a donor stem cell-derived protein in said sample;
wherein the presence of a donor stem cell-derived donor protein indicates that said stem cell transplant has grafted.

2. The method of claim 1, wherein said protein-containing sample is a body fluid sample.

3. The method of claim 2, wherein said protein-containing sample is a blood sample or a serum sample.

4. The method of claim 3, wherein said blood sample is a hematopoietic cell sample.

5. The method of claim 1, wherein identifying comprises multi-dimensional protein separation and mass spectrometry.

6. The method of claim 5, wherein multi-dimensional protein separation comprises HPLC, ion exchange and/or reverse phase chromatography.

7. The method of claim 6, wherein multi-dimensional protein separation comprises 2D-gel electrophoresis and isoelectric focusing electrophoresis.

8. The method of claim 1, further comprising obtaining a pre-transplant sample from said transplant recipient and characterizing proteins in said pre-transplant sample.

9. The method of claim 8, further comprising obtaining a protein-containing donor sample and characterizing stem cell-derived proteins from the donor.

10. The method of claim 9, wherein said protein-containing donor sample is a fluid, cell, tissue or organ sample.

11. The method of claim 1, wherein said donor is an allogeneic donor.

12. The method of claim 11, wherein said allogeneic donor has an HLA profile identical to said transplant recipient.

13. The method of claim 11, wherein said allogeneic donor has an HLA profile not identical to said transplant recipient.

14. The method of claim 1, wherein said donor is an xenogeneic donor.

15. The method of claim 1, wherein said transplant recipient is a mammal.

16. The method of claim 15, wherein said mammal is a human.

17. The method of claim 1, wherein said donor is a mammal

18. The method of claim 17, wherein said mammal is a human.

19. The method of claim 1, wherein step (a) is performed at a time sufficiently post-transplant that donor stem cell-derived proteins from ungrafted stem cells will not be present in said transplant recipient.

20. The method of claim 19, wherein step (a) is performed one week post transplant.

21. The method of claim 19, wherein step (a) is performed more than one week post transplant.

22. The method of claim 10, wherein said protein-containing cell is a embryonic stem cell, a hematopoietic stem cell, a neuronal stem cell, a bone marrow stem cell, a oral mucosa stem cell, epithelial stem cell, lung stem cell, skin stem cell, gut stem cell, liver stem cell, pancreas stem cell, islet cell stem cell, heart stem cell, muscle stem cell, vascular (endothelial) stem cell, kidney stem cell or mesenchymal stem cell.

23. The method of claim 10, wherein said protein-containing tissue is from the skin, the liver, the gastrointestinal tract, the kidney, the heart, the blood vessel or derived from the epithelial, mesodermal or endothelial organs.

24. A method of assessing stem cell differentiation following transplant comprising:
a) obtaining a protein-containing sample from a transplant recipient; and
b) identifying the presence or absence of a donor differentiated stem cell-derived protein in said sample;
wherein the presence of a donor differentiated stem cell-derived donor protein indicates that stem cell transplant has grafted and differentiated.

25. The method of claim 24, wherein the protein-containing sample is a body fluid sample.

26. The method of claim 25, wherein the protein-containing sample is a blood sample or a serum sample.

27. The method of claim 26, wherein the blood sample is a hematopoietic cell sample.

28. The method of claim 24, wherein identifying comprises multi-dimensional protein separation and mass spectrometry.

29. The method of claim 28, wherein multi-dimensional protein separation comprises HPLC, ion exchange and/or reverse phase chromatography.

30. The method of claim 29, wherein multi-dimensional protein separation comprises 2D-gel electrophoresis and isoelectric focusing electrophoresis.

31. The method of claim 24, further comprising obtaining a pre-transplant sample from said transplant recipient and characterizing proteins in said pre-transplant sample.

32. The method of claim 24, further comprising obtaining a protein-containing donor sample and characterizing stem cell-derived proteins from the donor.

33. The method of claim 32, wherein said protein-containing donor sample is a fluid, cell, tissue or organ sample.

34. The method of claim 24, wherein the donor is an allogeneic donor.

35. The method of claim 34, wherein the allogeneic donor has an HLA profile identical to said transplant recipient.

36. The method of claim 34, wherein the allogeneic donor has an HLA profile not identical to said transplant recipient.

37. The method of claim 24, wherein said donor is an xenogeneic donor.

38. The method of claim 24, wherein said transplant recipient is a mammal.

39. The method of claim 38, wherein said mammal is a human.

40. The method of claim 24, wherein said donor is a mammal

41. The method of claim 40, wherein said mammal is a human.

42. The method of claim 24, wherein step (a) is performed at a sufficient time post-transplant that differentiation of donor stem cells can occur.

43. The method of claim 42, wherein step (a) is performed one week post transplant.

44. The method of claim 42, wherein step (a) is performed more than one week post transplant.

45. The method of claim 33, wherein said protein-containing cell is a embryonic stem cell, a hematopoietic stem cell, a neuronal stem cell, a bone marrow stem cell, a oral mucosa stem cell, epithelial stem cell, lung stem cell, skin stem cell, gut stem cell, liver stem cell, pancreas stem cell, islet cell stem cell, heart stem cell, muscle stem cell, vascular (endothelial) stem cell, kidney stem cell or mesenchymal stem cell.

46. The method of claim 33, wherein said protein-containing tissue is from the skin, the liver, the gastrointestinal tract, the kidney, the heart, the blood vessel or derived from the epithelial, mesodermal or endothelial organs.

47. A method of determining tissue site engraftment of a stem cell comprising:
a) obtaining a sample from a post-transplant recipient; and
b) assessing said sample for the presence of a tissue selective donor-derived protein in said sample;
wherein the presence of a tissue selective donor-derived protein in said sample indicates that said stem cell has engrafted in a tissue site supporting expression of said tissue selective donor-derived protein.
